(19) United States
(12) Patent Application Publication
(10) Pub. No.: US 2007/0166702 A1
(43) Pub. Date: Jul. 19, 2007

(54) Methods for predicting the efficacy or outcome of hcv therapy

(76) Inventors: Geert Leroux-Roels, GENT (BE); Isabelle Desombere, Merelbeke (BE)

Correspondence Address: CLARK & ELBING LLP, 101 FEDERAL STREET, BOSTON, MA, 02110, US

(21) Appl. No.: 11/572,200

(22) PCT Filed: Jul. 18, 2005
(86) PCT No.: PCT/BE05/00118

§ 371 Date: Jan. 16, 2007

Related U.S. Application Data
(60) Provisional application No. 60/588,522, filed on Jul. 16, 2004.

(51) Int. Cl. C12Q 1/70

(52) U.S. Cl. 435/5

(57) ABSTRACT

The present invention relates to a novel method for predicting likeliness of the outcome or efficacy of an antiviral therapy against Hepatitis C Virus (HCV). This method is based on the detection of antibodies against the proteins NSta and or NS5a in a sample of a patient. This method can be further combined with the detection of viral titers of HCV in a sample of a patient. The method of the invention allows a more reliable identification of a subset of non-responders in an early stage of treatment.

### FIELD OF THE INVENTION

The invention relates to methods for predicting and monitoring the efficacy or outcome of therapies against Hepatitis C Virus (HCV). The invention further relates to diagnostics comprising peptides for antibody detection and diagnostics for the determination of viral titers.

### BACKGROUND OF THE INVENTION

The hepatitis C virus (HCV) infects more than 170 million people worldwide and represents an important public health problem. Approximately 30% of people infected with HCV clear the virus while 70% progress to chronic infection [Alter et al. (1999) N Engl J Med 341, 556-562]. The mechanisms determining clearance or persistence remain obscure. Recent data suggest that the concerted action of both the cellular and humoral arms of the adaptive immune response ultimately control the HCV infection. In addition, the innate immune system plays a role in protecting the host during the initial period of virus infection, and in shaping the nature of the adaptive immune response [Bertoletti & Ferrari (2003) Hepatology 38, 4-13].

It has been proposed that vigorous and multi-specific CD4 responses of T helper 1 nature and equally strong and multi-specific CD8 responses represent the main mechanisms responsible for HCV control, whereas a defect in these responses leads to chronicity [Lauer & Walker (2001) N Engl J Med 345, 41-52]. The lack of suitable animal models to study the pathogenesis of HCV infections has hampered the definitive demonstration of causal relationships between the different outcomes of infection and the vigour and breadth of T-cell responses. A recent report in HCV-infected chimpanzees wherein no clear association between T-cell response and virus clearance could be found, warrants caution in interpreting clinico-immunological findings [Thomson et al. (2003) J Virol 77, 862-870]. On the other hand, it is hard to imagine that the cellular immune response would play no role in HC-V control.

The importance of the humoral immune responses in controlling the chronic HCV infection has been demonstrated in HCV infected patients with agammaglobulinemia. These patients show a rapid progression of liver disease [Bjoro et al. (1994) N Engl J Med 331, 1607-1611]. Despite the fact that HCV is known for over a decade and despite the fact that thousands of infected patients have been carefully observed and monitored, many aspects of the humoral response are ill-defined and the estimate of the importance of the antibody response in viral clearance cannot be made. Certain experimental data suggest that HCV clearance is temporally associated with the production of neutralizing anti-envelope antibodies [Zibert et al. (1997) Hepatology 25, 1245-1249] and sera with high levels of anti-envelope antibodies can prevent HCV infections [Farci et al. (1996) Proc Natl Acad Sci USA 93, 15394-15399]. Other data, however, cast doubt on the role of these antibodies in the resolution of HCV infection [Prince et al. (1999) J Infect Dis 180, 987-991]. Cooper et al. [cited above] demonstrated that, although HCV-specific humoral responses are commonly seen in HCV-infected chimpanzees and humans, HCV-specific antibodies are not capable of conferring protection. Only a few studies have examined the possible role of antibodies to the non-structural (NS) proteins on viral clearance or the evolution of chronic infection and the results are inconclusive. One study revealed a broader antibody response in chronic HCV patients as compared to subjects who cleared the infection [Zhang et al. (1995) J Infect Dis 171, 1356-1359]. Overall, the different components of the adaptive immune system are so interconnected that the failure of one clearly affects the expansion and protective efficacy of the others, e.g. lack of CD4+ T-helper cells can impair both CD8+ T-cell activity and antibody production, while the inability to mount a virus-specific CD8+ T-cell response results in a level of circulating virus that cannot be cleared by antibodies alone [Kalams & Walker (1998) J Exp Med 188, 2199-2204; Ciurea et al. (2001) J Exp Med 193, 297-305].

A characteristic of the humoral immune response to HCV infection is the delayed appearance of anti-HCV antibodies. Apart from the antibody response to the HCV core protein, antibodies to all other proteins rarely appear before month 6 of infection, a time point at which chronicity has been established. Chen et al. [Chen et al. (1999) Gastroenterology 116, 135-143] demonstrated a quantitatively (low serum antibody levels) and qualitatively (IgG1-restricted) limited humoral immune response to HCV infection that develops rather slowly during the transition from acute to chronic infection. Nikolaeva et al. [ (2002) J Viral Hepat 9, 429437] demonstrated that the titer values of IgG antibodies against NS proteins were higher in patients with a natural course of chronic HCV infection than in patients with acute and resolving HCV infection.

Beld et al. [Hepatology 29, 1288-1298] reported that apparent viral clearance from blood was associated with a significant decrease of antibodies to NS3 and NS5 proteins in injecting drug users. Bassett et al. [(1998) J Virol 72, 2589-2599] demonstrated that HCV-inoculated chimpanzees which cleared the virus, displayed poor responses against NS3 and NS4 proteins and rapidly lost antibodies against NS5 protein.

Because of the physical, psychological and financial burden they impose on patients, IFN-based therapies should ideally only be administered to those patients in whom they induce a sustained response, the so-called sustained responders (SR). Despite the fact that certain baseline factors predicting SR have been defined, none of these turns out to be flawless and additional or better markers are still needed. The best independent pre-treatment characteristics known to be associated with an increased chance for SR are: genotype 2 or 3 and low baseline viral load [Berg et al. (2003) Hepatology 37, 600-609; Strader et al. (2004) Hepatology 39, 1147-1171; Davis (2002) Hepatology 36, S145-151; Gao et al. (2004) Hepatology 39, 880-890]. However, even in the most favourable population where all positive predictive characteristics are present, the rates of virological SR with standard combination therapy reached only 79% [Poynard et al. (2002) Hepatology 31, 2 11-218] and an exact prediction of response to treatment for an individual patient was not possible on the basis of these pre-treatment parameters [Knolle et al. (1998) J Viral Hepat 5, 399-406]. Rather than focusing on pre-treatment characteristics, guidelines for continuation or cessation of IFN-based regimens are now focusing on the evolution of the viral load after three months of therapy [Strader et al. (2004) Hepatology 39, 1147-1171; Keeffe et al. (2002) Rev Gastroenterol Disord 2, 34-40]. In several trials, it was demonstrated that a strong HCV-RNA decrease during the first weeks of therapy (=EVR, early virological response) can be used to predict long-term therapeutic response. A fall in viral load to undetectable or by at least 2 log₁₀ units after 12 weeks was found to be the optimal definition of an EVR. Furthermore, all patients with detectable HCV-RNA levels in serum after 24 weeks of therapy became virologic non-responders (NR) [McHutchison et al. (1998) N Engl J Med 339, 1485-1492; Poynard et al. (1998) Lancet 352, 1426-1432] Because immunoassays to monitor humoral responses to individual HCV proteins are not routinely used in medical practice, antibody responses against individual HCV proteins have been poorly investigated and little is known about their predictive value on the outcome of disease or response to therapy.

### SUMMARY OF THE INVENTION

According to a first aspect the invention relates to a kit, comprising one or more HCV antigenic peptides, each of said one or more HCV antigenic peptides being selected from a) HCV antigenic peptides comprising in its sequence at least 8 amino acids of the NS4a protein of HCV and/or b) HCV antigenic peptides comprising in its sequence at least 8 amino acids of the NS5a protein sequence of HCV. Thus, according to the present invention the HCV antigenic peptides provided are only the ones selected from (a) and/or (b) and not any other HCV antigens.

According to a particular embodiment of the invention, the kit can be used in predicting the outcome or efficacy of a therapy against Hepatitis C Virus (HCV) based on interferon alpha.

According to a further particular embodiment, the invention provides a kit consisting of one or more peptides having in their sequence at least 8 amino acids of the NS4a protein of HCV and/or one or more peptides comprising in their sequence at least 8 amino acids of the NS5a protein sequence of HCV.

According to a further particular embodiment a kit comprising a) one or more peptides comprising in their sequence at least 8 amino acids of the NS4a protein of HCV and/or b) one or more peptides comprising in their sequence at least 8 amino acids of the NS5a protein sequence of HCV, and further comprising one or more reagents suitable for determining active HCV infection in a sample of a patient. More particularly, according to the present invention such reagents suitable for determining active HCV infection in a sample includes primers for the detection of HCV RNA levels for simultaneous or sequential testing and/or antibodies for the selective detection and quantification of HCV core antigen for simultaneous or sequential testing. According to a particular embodiment such a kit is a diagnostic kit for predicting the outcome of a therapy against Hepatitis C Virus (HCV) based on interferon alpha.

According to one embodiment, the kit of the invention comprises the one or more peptides of the NS4a protein sequence and/or the one or more peptides of the NS5a protein sequence physically separated from each other.

According to one embodiment the one or more peptides of the NS4a protein sequence are peptides comprising in their sequence at least 8 amino acids, most particularly 8 consecutive amino acids of SEQ ID NO: 1 or SEQ ID NO: 2 or are peptides comprising in their sequence the sequence of SEQ ID NO: 3. Alternatively the one or more peptides of the NS4a protein sequence have the sequence of SEQ ID NO: 1 and/or SEQ ID NO: 2.

According to another embodiment of the invention the one or more peptides of the NS4a protein sequence present in the kit of the invention is/are peptides comprising in their sequence at least 8 amino acids, most particularly 8 consecutive amino acids of SEQ ID NO: 7 or SEQ ID NO: 8 or comprising in their sequence the sequence of SEQ ID NO: 9. Alternatively the one or more peptides of the NS4a protein sequence present in the kit of the invention consist of the sequence represented by SEQ ID NO: 7 or SEQ ID NO: 8.

In an alternative embodiment the peptides present in the kit of the invention are peptides which comprise 8 consecutive amino acids corresponding to a sequence within the partial NS5a sequence spanning the 2209-2274 region (ISDR domain), the 2250-2300 region (PKR binding domain) or the 2275-2321 region (region between the PKR binding domain and the basal phosphorylation region). The one or more peptides corresponding to a sequence within the NS5a protein sequence are, according to one embodiment, peptides comprising in their sequence at least 8 consecutive amino acids of SEQ ID NO: 4 or SEQ ID NO: 5. Alternatively or additionally, the one or more peptides of the NS5a protein sequence present in the kit of the invention are peptides comprising the sequence of SEQ ID NO: 6. Alternatively, the one or more peptides of the NS5a protein sequence are represented by SEQ ID NO: 4 or SEQ ID NO: 5. In a particular embodiment the one or more peptides present in the kit of the invention include one or more peptides with sequences overlapping the peptide sequences recited above.

Further alternative embodiments of the present invention include kits wherein the one or more peptides of the NS5a protein sequence are peptides comprising in their sequence at least 8 amino acids, more particularly 8 consecutive amino acids of SEQ ID NO: 10 or SEQ ID NO: 11. The one or more peptides of the NS5a protein sequence present in the kit of the invention include peptides comprising the sequence of SEQ ID NO: 12. Alternatively, the one or more peptides of the NS5a protein sequence are represented by SEQ ID NO: 10 or SEQ ID NO: 12. In a particular embodiment the one or more peptides present in the kit of the invention include one or more peptides with sequences overlapping the peptide sequences recited above.

Another aspect of the invention relates to an in vitro method for predicting the efficacy or outcome of a therapy against Hepatitis C Virus (HCV) based on interferon alpha said method comprising the steps of determining the level of binding of antibodies in a tissue sample (commonly used are serum or plasma) of an HCV infected patient with one or more peptides comprising in their sequence at least 8 (consecutive) amino acids of the NS4a protein of HCV and/or one or more peptides comprising in their sequence at least 8 amino acids of the NS5a protein sequence of HCV. This method of determining the level of binding can be quantitative.

In this method, the one or more peptides corresponding to the NS4a protein sequence can be peptides comprising in their sequence at least 8 (consecutive) amino acids of SEQ ID NO: 1, SEQ ID NO: 2, SEQ ID NO: 7 or SEQ ID NO: 8. Alternatively, the one or more peptides of said part of the NS4a protein sequence can be peptides comprising the sequence of SEQ ID NO: 3 and/or SEQ ID NO: 9. Alternatively, one or more peptides of said part of the NS4a protein sequence can be represented by SEQ ID NO: 1, SEQ ID NO: 2, SEQ ID NO: 7 and/or SEQ ID NO: 8.

According to a particular embodiment the method includes detection of antibodies against NS5a protein. More particularly, the method of the invention can include the detection using one or more peptides of the NS5a protein sequence which can be peptides comprising the sequence of SEQ ID NO: 6 and/or SEQ ID NO: 12. Alternatively said one or more peptides of said part of the NS5a protein sequence can be represented by SEQ ID NO: 4, SEQ ID NO: 5, SEQ ID NO: 10 and/ or SEQ ID NO: 11.

According to another particular embodiment, the method of the invention includes detection of antibodies against particular regions of the NS5a protein, such as the 2209-2274 region, the 2250-2300 region and/or the 2275-2321 region. Most particularly, the one or more peptides of the NS5a protein sequence can be peptides comprising in their sequence at least 8 (consecutive) amino acids of SEQ ID NO: 4 or SEQ ID NO: 5, SEQ ID NO: 10 and/or SEQ ID NO: 11. The one or more peptides which are used in the method of the invention can be peptides with sequences overlapping the sequences recited above.

In addition the method can further comprise the step of detecting active infection in the patient. More particularly detection of active infection can be performed by detecting the level of HCV RNA in a tissue sample (e.g. serum or plasma), or can comprise the step of detecting a level of HCV core antigen in a tissue sample (e.g. serum or plasma) or the level of another compound which concentration is correlated with the concentration of HCV RNA.

The method can be performed on a sample taken after about at least 4 days, preferably at least 6 days after the start of HCV therapy of the patient.

According to one aspect of the invention, the method is used to predict efficacy of treatment, more particularly to predict a non-responding outcome, based on the presence of antibodies below a particular threshold. More particularly the method can involve determining whether the level of binding of antibodies to the HCV antigenic peptides (e.g.) prior to the start of treatment is below a threshold value of 50, 10, 5 or even 1 and, optionally additionally determining that the level of HCV RNA in a sample of that same patient during treatment is above 105 IU/ml, and determining that predicted outcome is that of non-response based thereon. According to a further embodiment this method is used to predict a non-responding outcome, based on a level of binding (prior to treatment of the patient) of antibodies to the above described HCV antigenic peptides which is below a threshold value of 50, 10, 5 or even 1 and, optionally additionally based on a level of HCV core antigen determined in a sample of the same patient taken during treatment to be above 15 pg/ml.

According to a particular embodiment of the above-described method, the threshold level of binding of antibodies to the HCV antigenic peptides of the invention, used to determine whether or not the treatment will be effective, is 1. This means that even in an undiluted sample, no binding is determined (being below the sensitivity of the assay) with the NS4a and/or NS5a HCV antigenic peptides of the present invention. Depending on the peptides used, the method can be used for any genotype of HCV. According to a particular embodiment HCV is of genotype 1.

Other aspects of the invention relate to isolated peptides with a length between 8 and 30 amino acids comprising a sequence of at least 8 (consecutive) amino acids of a sequence corresponding to SEQ ID NO: 1, 2, 4, 5, 7, 8, 10, and/or 11. The invention also relates to one or more isolated peptides being selected from the group represented by SEQ ID NO: 1, SEQ ID NO: 2, SEQ ID NO: 4, SEQ ID NO: 5. The invention also relates to an isolated peptide with a length between 8 and 30 amino acids comprising the sequence represented by SEQ ID NO: 4, SEQ ID NO: 6, SEQ ID NO: 9 or SEQ ID NO: 12. The invention also relates to one or more isolated peptides being selected from the group represented by SEQ ID NO: 3, SEQ ID NO: 6, SEQ ID NO: 9 and SEQ ID NO: 12.

In one embodiment the present invention relates to peptides between 8 and 30 amino acids comprising in their sequence at least 8 (consecutive) amino acids of HCV NS4a or NS5a protein sequence. The NS4a or NS5a protein sequence can be a sequence of any isolate, strain or genotype of HCV. At the carboxyterminal or aminoterminal side of the peptide comprising the at least 8 consecutive amino acids as defined above is optionally a peptide with a sequence identical or similar (90, 80, 70 or 60% identical) or unrelated to NS4a or NS5a, for example a sequence comprising a spacer for attaching the peptide to a support. In particular embodiments, NS4a derived peptides can be used comprising the sequence VLYREFDE [SEQ ID NO: 3] or a sequence derived thereof represented by the motif [VLI]-L-Y-[RQE]-[EAQ]-[FY]-D-E. This motif also comprises those sequences which are encountered in different genotypes of HCV. In other particular embodiments, NS4a derived peptides are used comprising the sequence LSGKPAIIPDREVLYREFDE [SEQ ID NO:1]) or VLYREFDEMEECSQHLPYIE [SEQ ID NO: 2] or sequences derived thereof represented by the motif [LVI]-[SNEGT]-[GQD]-[KRQ]-[PATV]-[AV]-[IVL]-[IVAT]-P-D-[RK]-[EQ]-[VLI]-L-Y-[RQE]-[EAQ]-[FY]-D-E or [VLI]-L-Y-[RQE]-[AEQ]-[FY]-D-E-M-E-E-C-[SA]-[QSKA]-[HRKAS]-[LAI]-[PA]-[YL]-[IVLM]-[EDA]. These motifs also comprise those sequences which are encountered in different genotypes of HCV.

In another embodiment the present invention relates to peptides between 8 and 30 amino acids being similar to a fragment of NS4a or NS5a of any strain, isolate or genotype of HCV (the similarity can be expressed as a percentage of identity between two sequences and can range from about 70%, 75%, 80%, 85, 90, to 95 or 96%).

A further aspect of the present invention relates to strategies for determining the treatment of a patient having been diagnosed with HCV, which strategies include using the methods of the present invention to determine whether or not to start or continue treatment of the patient with interferon-alpha based treatments.

Yet another aspect of the present invention relates to methods of manufacturing a diagnostic tool, which method comprises combining one or more HCV antigenic peptides of NS4a and/or NS5a and optionally one or more reagents which can be used to determine the presence of HCV particles (viral titre) and optionally suitable standard kit reagents. Optionally the kit of the present invention comprises a paper insert describing the stopping rule described herein and the method of manufacture comprises adding the paper insert.

### BRIEF DESCRIPTION OF THE DRAWINGS

FIG. 1 described the magnitude of antibody responses to HCV specific non-structural (NS) proteins in population 1 of chronic HCV patients. For anti-NS4a and anti-NS5a antibodies, 62 and 60 SR (sustained responders) were compared to 62 and 54 NR (non-responders), respectively. Antibody titers are endpoint dilutions as measured in EIA. P-values were calculated with the Mann Whitney U test. In the Box-and-whisker plots, the central box represents the values from the lower to upper quartile. The middle line represents the median.

FIG. 2 describes a proposed algorithm for management of patients with chronic genotype 1 positive hepatitis C during antiviral therapy. The baseline HCV-RNA quantitative measurement can be performed retrospectively on a frozen serum-sample.

### GLOSSARY

The following abbreviations are used in the present application: HCV, hepatitis C virus; NS, non-structural; SR, sustained response; NR, non-response; IFN-alpha, interferon-alpha; ALT, alanine aminotransferase; REL, relapsers; TMA, transcription-mediated amplification; AA, amino acid; EIA, enzyme immunoassay; LIA, Line immunoassay; RR, relative risk; PPV, positive prediction value; NPV, negative prediction value.

Definitions

“NS4a” and “NS5a” as used herein refers to two of the Non-Structural proteins of the Hepatitis C virus. Several variants of these proteins having differences in their amino acid sequence have been identified. In a particular embodiment of the inventions NS4a and NS5a have an amino acid sequence as encountered in the HCV genotype 1.

The term “HCV antigenic peptide” as used herein refers to a peptide comprising a sequence which corresponds to part of a sequence of an antigen of the HCV virus, i.e. a protein which is produced by HCV and elicits an immune response. Antigens of the HCV virus particularly envisaged in the context of the present invention are NS4a and NS5a and the HCV antigenic peptides comprising sequences corresponding to these HCV antigens are referred to herein as ‘NS4a antigenic peptide’ and ‘NS5a antigenic peptide’, respectively.

The phrase “predicting the efficacy or outcome of a HCV therapy” as used in the present application refers to determining with a certain statistical certainty whether a HCV infected patient (either prior to or during antiviral treatment, most particularly treatment based on interferon alpha) can potentially benefit from such therapy.

The term “baseline” in the present invention refers to a time point or a condition prior to or at the beginning of the antiviral therapy.

The phrase “presence or absence of NS4a or NS5a antibodies” in a sample as used herein, refers to an amount of antibodies over a certain threshold is observed. Such a threshold value can be expressed as endpoint dilutions, i.e. the dilution at which the lower limit of detection is achieved. Thus a threshold of 50 means that at a 50-fold dilution of the sample the limit of detection is achieved, a threshold of 1 refers to the fact that the amount of antibodies present in the sample is such that the (bottom) limit of detection is reached in a undiluted sample. Patients for which antibodies against NS4a and/or NS5a are or are not detected prior to treatment will also be referred to as NS4a/NS5a positive or negative patients, respectively.

### DETAILED DESCRIPTION OF THE INVENTION

The present invention relates to new predictive baseline parameters in the treatment of HCV. Since the baseline antibody levels to one or more HCV proteins could be a marker of the quality of the patient's immune response towards the virus, the present invention makes use of antibody levels for predicting the outcome of therapy. In the context of the present invention therapy refers to antiviral therapy, more particularly antiviral therapies dedicated to boost the protective host response to viruses, most particularly antiviral therapies based on interferon, such as, but not limited to therapies which include the administration of interferon alpha (including naturally or non-naturally occurring versions of interferon-alpha 2a or 2b, either pegylated or non-pegylated forms thereof) and/or beta interferons and/or combinations thereof optionally with other antiviral agents such as, but not limited to the combination of one of the above-mentioned types of interferon with ribavirin.

Basis for the present invention was an investigation into the occurrence and serum levels of IgG against HCV NS proteins at the onset of therapy in a population of genotype 1 positive chronic HCV-patients. The prevalence and serum-levels of these antibodies were correlated with treatment outcome.

Different therapeutic outcomes can be observed namely: 1) sustained responders (SR) 6 months after therapy; 2) relapsers after therapy (REL); 3) breakthrough during therapy; 4) non-responders after therapy (NR). In a particular embodiment the prediction of the therapy is the prediction whether a person about to be treated or being treated with antiviral therapy, more particularly treatments based on interferon alpha, is likely to benefit from such therapy or not. More particularly, the combination of detection of antibodies and HCV RNA detection allows the identification of patients as relapsers after therapy (REL) or non-responders after therapy (NR), also being denominated as non-sustained responder (NSR), at a much earlier stage than classical methods. Detecting, at an early stage of the therapy, persons which will not benefit from an interferon based HCV therapy, allows deciding whether or not to stop the therapy. This decision to stop at an early stage of the therapy is further referred to as the “early stopping rule”.

The present invention is based on the observation that there is a correlation between the presence of antibodies against NS4a and/or NS5a in serum samples prior to therapy and the non-responsiveness to antiviral therapy. Measured antibody levels against NS3, NS4a and NS5a were determined at baseline (prior to therapy) in the serum of 245 patients that were chronically infected with HCV of genotype 1. Careful clinical and virological monitoring allowed the classification of these patients as sustained responders (SR), relapsers (REL), non-responders (NR) or non-sustained responders (REL+NR) to therapy. While no differences were observed in the prevalence and magnitude of the anti-NS3 responses in these different response groups, anti-NS4a and anti-NS5a antibodies were observed more frequently and at higher titers in SR versus NR or NSR. In one aspect of the present invention these antibody tests, alone or in combination, are used to predict the likelihood that the antiviral therapy has a positive outcome. Thus, the present invention provides methods for predicting the efficacy or outcome of HCV therapy. Based on the presence or absence of antibodies alone, it is estimated that it can be predicted whether or not the patient can benefit from therapy with a statistical certainty of between 75-100%.

It has moreover been found that while the simultaneous absence of antibodies to NS4a and NS5a was not superior in predicting non-sustained response than the absence of each antibody alone, a test combination consisting of determining the absence of NS4a (and/or NS5a) antibody at baseline together with determining the presence of HCV RNA exceeding 10⁵ IU/l after one week of treatment provides a positive predictive value for non-sustained response to treatment of 100% (with 100% statistical certainty). Alternatively, instead of determining HCV RNA it is also possible to detect HCV core antigen. In this case, the combination of NS4a (and/or NS5a) antibody detection an the presence of HCV core antigen exceeding 15 pg/ml after one week of treatment, provides a positive predictive value for non-sustained response to treatment of 100%. This diagnostic algorithm for management of patients with chronic HCV improves existing ‘stop rules’ (i.e. a decision to end an unsuccessful therapy), by allowing the identification of non-sustained responders after one week and leads to additional cost reductions.

To exclude any genotype-based bias, only genotype 1 positive patients were examined. Based on the assumption that the humoral status of the patient at the onset of therapy may have a predictive value, IgG antibodies were qualified against HCV NS proteins at baseline in a population of chronic HCV-patients. While the occurrence and the titers of anti-NS3 antibodies did not differ between SR and NR, the presence of anti-NS4a and anti-NS5a antibodies was significantly correlated with the response to combination therapy.

As with most biological parameters, however, the presence of these antibodies in SR and their absence in NR is not absolute. At the start of therapy, 87%, and 82% of the SR, and 48% and 44% of the NR had antibodies to NS4a and NS5a, respectively. Not only the prevalence of these antibodies, but also the magnitude of these antibody responses was strongly correlated (p<0,001) with response to therapy.

In one aspect, the present invention defines one parameter or a set of parameters that allows the identification of NSR (=NR+REL) as early as possible in the treatment course, or in other words the creation of an ‘early stopping rule’. The decision is ideally taken before 12 weeks, preferably before or at about 6 weeks, more preferably before or at about 2 weeks and even more preferably before or at about 8, 7, 6, 5 or 4 days after the start of therapy. The definition of this stopping rule parameter thus relies on maximizing the PPV for NSR, i.e. minimizing discontinuation of treatment of patients who might ultimately benefit after completion of the full course of therapy. In the present study, the PPV for NSR for the absence of antibodies to NS4a or anti-NS5a at baseline was about 73 to 87%, depending on the population studied. In conventional techniques the maximum NPV for SR of a baseline parameter was 73% for low gamma-glutamyltransferase levels and genotype 1. The absence of anti-NS4a or anti-NS5a at baseline as shown by the present invention is superior, or at least as useful as a pre-treatment predictor for NSR.

Since the loss of potential responders should be as low as possible, a PPV for NSR of 87% is satisfactory as it is better than achieved previously, however an improvement is preferred. The simultaneous absence of both anti-NSA4 and anti-NS5a antibodies did not improve the predictive value for NSR. The predictive value was improved in accordance with an embodiment of the present invention by combining the absence of antibodies with one or more different measurements of HCV infective agents, such as HCV-RNA or HCV-antigen measurements. In accordance with an embodiment of the present invention an HCV-RNA level after one week of treatment exceeding 100 000 IU/ml, combined with the absence of anti-NS4a or anti-NS5a antibodies at baseline had a PPV for NSR of 100%. So, using this test combination as a basis for implementing a stopping rule allows the early identification of a subset of non-sustained responders, while treatment will not be stopped in any patient that might ultimately respond after completion of the full course of therapy. Since an HCV-RNA level at treatment week 1 above 100 000 IU/ml alone has only a predictive value for NSR of 87,7%, it is the synergistic combination with the pre-treatment antibody profile which generates the strength of this ‘early stopping rule’, i.e. identification before start of treatment of the presence of antibodies to NS4a or NS5a allows the identification of patients which, based on HCV-RNA level at one week would be discontinued from treatment, while in fact being potential sustained responders. Thus, in accordance with an embodiment of the present invention the prediction of whether or not antiviral therapy is expected to be successful is improved by a quantitative HCV RNA measurement, e.g. performed after about 1 week of therapy. The additional cost induced by this test is amply compensated by the savings on treatment costs by early discontinuation of treatment in those patients who can be predicted, with 100% accuracy not to benefit from antiviral therapy, i.e. those patients that do not display anti NS4a/NS5a antibodies in their serum prior to treatment and have HCV RNA titers above threshold after 1 week of therapy. If this stopping rule is applied, 1/3 of NSR patients would discontinue treatment at week 1 (=sensitivity of 28-39%), resulting in 33% cost savings of total costs of treating all NSR patients with a full course. Early discontinuation of therapy at week 12 according to the level 2 stopping rule of the prior art (i.e. 2 log₁₀ HCV-RNA decline at week 12) saves 30% of the costs of treating all NSR with a full course. Since stopping rules are designed to minimise discontinuation of treatment in patients who might ultimately respond, diagnostic specificity is given priority on diagnostic sensitivity. Reducing the specificity will increase the sensitivity and will thus lead to greater cost savings. The consequence of this is that patients who would have cleared the virus would be discontinued, which would ultimately lead to more individual suffering and societal costs.

The humoral responses against NS4a and NS5a in SR may be indirect markers of the quality of the cellular immune response to HCV. Assuming that the humoral immune response to NS proteins is normally short-lived, the continuous presence of antibodies to NS4a and/or NS5a may be a marker of ongoing, possibly immune-mediated, cell destruction. In this view the absence of anti-NS4a/anti-NS5a can be considered as a sign of weak or absent immune responses, while the presence of these antibodies points towards an active, ongoing immune response.

The present invention relates to kits and methods for use in predicting the efficacy or outcome of HCV treatment, based on the detection in a sample of a patient of antibodies against the HCV antigens NS4a and/or NS5a.

According to a particular embodiment of the invention, the detection of antibodies is ensured by an assay which detects the binding of the NS4a and/or NS5a antibodies to HCV antigenic peptides. According to the present invention such an HCV antigenic peptide is a peptide comprising a sequence corresponding to part of an HCV antigen, more particularly corresponding to part of the NS4a and/or NS5a antigens. Indeed, the method and kits designed for the purpose of predicting the efficacy of HCV treatment according to the invention are particularly characterized in that they are based on the detection of antibodies specifically against NS4a and/or NS5a only and do not necessarily involve the detection of antigens against other HCV antigens. Thus, though the methods and kits of the present invention may involve other reagents, the HCV antigenic peptides used for predicting the outcome of HCV therapy only include peptides which comprise a sequence corresponding to the NS4a and/or NS5a proteins. This is different from the existing assays used for screening for HCV infection, which are based on a wide variety of antigens, which are moreover mixed.

According to a particular embodiment the HCV antigenic peptides used in the context of the invention are peptides, more particularly but not necessarily limited to peptides of between 8 and 30 amino acids, comprising in their sequence at least 8 amino acids of HCV NS4a (referred to as ‘NS4a antigenic peptide’) or NS5a (referred to as ‘NS4a antigenic peptide’) protein sequence. Most particularly said 8 amino acids correspond to 8 consecutive amino acids in the NS4a or NS5a protein sequence. A sequence corresponding to a sequence of the NS4a or NS5a HCV antigen protein sequence according to the present invention is a sequence which is immunologically identical (i.e. elicits the same immune response in humans) as the sequence observed in the NS4a or NS5a protein. According to a particular embodiment such a sequence is a sequence which is identical to a sequence of the NS4a or NS5a protein. It will however be understood to the skilled person that minor variations may be introduced into this sequence without affecting the immunologic identity to NS4a or NS5a. Thus, according to a particular embodiment of the present invention the HCV antigenic peptides comprise a sequence of at least 8, particularly between 8 and 30 consecutive amino acids which is essentially similar to a sequence of the NS4a or NS5a HCV antigen. The similarity can be expressed as a percentage of identity between two sequences when optimally aligned and can range from about 70%, 75%, 80%, 85, 90, to 95 or 96%). The NS4a or NS5a protein sequences referred to in the context of the present invention include the sequence of NS4a and NS5a of any isolate, strain or genotype of HCV. Differences which for instance can be tolerated between the sequence of the HCV antigenic peptide of the invention and the NS4a or NS5a antigen are difference which correspond to inter-isolate, inter-strain or inter-genotype variations. According to a particular embodiment, the HCV antigenic sequence(s) comprise a sequence corresponding to the sequence of the NS4a and/or NS5a protein of genotype Ia or Ib.

The HCV antigenic peptides of the present invention may moreover comprise additional sequences. Thus, for instance, according to particular embodiments of the present invention, the HCV antigenic peptides comprise at the carboxyterminal or aminoterminal side of the sequence of at least 8 (consecutive) amino acids a peptide with a sequence identical or similar (90, 80, 70 or 60% identical) or a peptide which is unrelated to NS4a or NS5a, for example a spacer for attaching the peptide to a support.

The methods and kits of the present invention relate to the detection of antibodies in a sample which bind to the HCV antigenic peptide(s) described herein. Detecting the binding of an antibody present in a sample to a particular antigen can be done in a number of different ways, known to the skilled person. One particular embodiment of the present invention involves the use of HCV antigenic peptides bound to a solid support (also referred to as solid-phase antigens). According to this embodiment, antibodies present in the sample which recognize the solid phase antigen will bind and the binding of these antibodies can be detected using either a labelled secondary antibody (such as, but not limited to goat-, rabbit-, mouse-anti-Human IgG) or a secondary antibody and a labelled tertiary antibody (e.g. rabbit-anti-mouse IgG etc.). Alternatively, detection of the binding of an antibody present in a sample to an antigen is ensured by labelling the antigen. Different embodiments of this technique are known to the skilled person and include but are not limited to methods which involve the binding of IgG present in the sample to a support and detection of labelled antigen bound thereto, co-precipitation methods etc . . .

According to a particular embodiment, the HCV antigenic peptides of the present invention are attached to a support via covalent bonds or non-covalent interactions such that the peptide(s) become(s) immobilised. The solid support can be nitrocellulose, polystyrene, nylon or any other natural or synthetic polymer. The HCV antigenic peptides of the present invention can also be passively adsorbed to plastics as described in the examples section.

Alternatively the HCV antigenic peptides of the present invention can be biotinylated peptides, for example as streptavidin-biotinylated peptide complexes or avidin-biotinylated peptide complexes. In the coupling, such as a complex of streptavidin-biotinylated peptides or avidin-biotinylated peptides, the peptides may be coupled either N-terminally, C-terminally or internally.

As indicated above, the present invention relates to methods and kits based on the detection of antibodies in a sample to antigenic peptides described herein which can be either or both NS4a antigenic peptides and/or NS5a antigenic peptides. According to a particular embodiment of the invention the method or kit involves the detection of antibodies to both NS4a and NS5a antigenic peptides. Alternatively, the detection of antibodies binding to each of these types of antigenic peptides is ensured separately. In any case, the detection of antibodies to either or both of the NS4a and/or NS5a antigen is ensured separately from the detection of any other antigens. This can be done in different ways, depending on the detection method used. According to a particular embodiment of the invention, the detection of antibodies binding to the one or more HCV antigenic peptide(s) of the invention is ensured by binding of the HCV antigenic peptide(s) to a solid support and detecting the binding of antibodies thereto with a secondary antibody and the separate detection of anti-NS4a and anti-NS5a antibodies can be ensured by physically separating the NS4a and NS5a antigenic peptides of the invention on the solid support. By “physically separated” refers to peptides which are spotted on, or coupled or attached to different places of a support or spotted on, coupled or attached to a different support, in order to allow separate detection of the binding to the peptides. Separate detection makes it possible to determine qualitatively, semi-quantitatively or quantitatively the relative contribution of the binding of antibodies against the different peptides, such as the NS4a and NS5a antigenic peptide(s).

Alternatively, the detection of antibodies binding to one ore more HCV antigens is ensured by use of labelled HCV antigenic peptides described above and the differential detection of anti-NS4a and anti-NS5a antibodies is ensured by differentially labelling the HCV antigenic peptides.

The methods and kits of the present invention may, besides the HCV antigenic peptides described herein, involve the use of other reagents and/or components. More particularly, according to a particular embodiment of the invention, detection of binding of antibodies in a sample to the HCV antigenic peptides of the invention is ensured by way of a secondary antibody, such as an anti-human IgG, more particularly a labelled anti-human IgG. Different types of

Additional reagents which are optionally used in the methods of the invention and optionally included in the kits of the invention include, but are not limited to positive and/or negative controls. A particular example of a positive control in the context of the present invention is normal human serum to which antibodies directed to the HCV antigenic peptide included in the method (i.e. the NS4a and NS5a antigens) have been added. A particular example of a negative control in the context of the present invention is human serum.

Further additional reagents which are optionally used in the methods of the invention and optionally included in the kits of the invention include, but are not limited to diluents (e.g. sample and secondary antibody diluents). Suitable examples of diluents which can be used in the context of the present invention are known to the skilled person and include phosphate buffers.

Further additional reagents which are optionally used in the methods of the invention and optionally included in the kits of the invention include reagents such as but not limited to substrate buffers which are required when the detection of the antibodies is based on enzymatic detection and wash buffers which can be used in the washing step. The substrate buffers for use in the context of the present invention will be determined by the enzyme used in the detection method (e.g peroxidase) and are known to the skilled person. Similarly, appropriate washing buffers for use in the context of the present invention are known to the skilled person and include, but are not limited to, phosphate buffers).

As indicated above, according to a particular embodiment, the methods and kits of the present invention comprise the detection of antibodies against one or more HCV antigenic peptides, whereby the antigenic peptide is bound to a solid support. Thus, a particular embodiment of the kit of the present invention comprises the HCV antigenic peptides of the present invention bound to a solid support, such as, but not limited to a microtiter plate with wells coated with the HCV antigenic peptides. Optional method steps can include coating the solid support with the HCV antigenic peptides of the invention.

According to a particular further embodiment, the methods and kits of the present invention further involve the detection of other parameters within the sample. Accordingly, the kits of the invention can comprise additional reagents suitable for this purpose.

More particularly, according to a particular embodiment, the methods and kits of the invention additionally involve the general detection of HCV antibodies in the sample. Indeed, the method of the invention can include components which are generally used to diagnose HCV based on HCV antibodies. In order to facilitate detection of all HCV antibodies in the sample, the kit can further contain a set of peptides for the detection of HCV in general (e.g. any protein of HCV or part thereof). According to one embodiment, the peptides are arranged in a way that the specific contribution of either antibodies against the NS4a or NS5a protein can be specifically detected. Alternatively, the methods and kits of the invention additionally involve the independent determination of total HCV antibodies, and suitable reagents for this purpose include but are not limited to a mixture of various HCV antigens.

Additionally or alternatively, the methods and kits of the present invention include steps and reagents respectfully related to the detection of HCV RNA in a sample. Indeed, according to a particular embodiment of the invention the detection of HCV RNA is performed on a sample taken at the same time-point as the sample used for the detection of antibodies to HCV antigens, e.g. before treatment, and is used to determine whether there is an active infection. Indeed, as the diagnosis of HCV may have been performed based on the presence of antibodies to total HCV antigens (using kits available in the art, such as, but not limited to the Innotest HCV ABIII, Innogenetics, Belgium) and/or a physical examination, the determination of an active viral infection requiring antiviral therapy can only be confirmed by the detection of viral antigens or RNA in the blood. Thus, according to a particular embodiment the methods of the present invention for predicting the outcome of HCV therapy include both detection of HCV RNA and detection of NS4a and/or NS5a reacting antibodies in sample(s) of a patient prior to the start of antiviral treatment, more particularly prior to the start of treatment with Interferon alpha. Additionally or alternatively, the combined use of detection of NS4a and/or NS5a reacting antibodies and detection of HCV RNA allows the accurate prediction of efficacy of therapy of a patient for HCV treatment. Most particularly it allows the prediction and/or identification of non-responders (NR), i.e. patients which do not respond positively to therapy with the antiviral agent, within one week of therapy. More particularly, according to a particular embodiment of the present invention, those patients in which no NS4a and/or NS5a reacting antibodies are detected prior to treatment are tested for the presence of HCV RNA after about one week of treatment. Presence of HCV RNA over a certain threshold amount, more particularly presence of more than 10exp5 RNA IU/ml is then considered as indicative of lack of efficacy of the treatment (=non-responder). Thus, according to this embodiment, depending on the outcome of the determination of NS4a and/or NS5a antibodies prior to treatment of the patient, an additional step is included to determine HCV RNA in a sample of this patient after about one week of antiviral treatment. Suitable method steps and reagents for detecting HCV RNA in a sample are known to the skilled person and illustrated in the Example section herein. Particular embodiments of additional reagents for determining HCV RNA include PCR primers for use in the specific detection of HCV RNA.

Additionally or alternatively, the methods and kits of the present invention include the detection of the level of HCV core antigen in the sample. Suitable method steps and reagents for detecting Core antigen in a sample are known to the skilled person and illustrated in the Example section herein. Alternative embodiments of the methods and kits of the invention are methods and kits which further comprise respectively steps and reagents suitable for the detection of an additional component in the sample of which the concentration is correlated with the concentration of HCV in the sample.

The present invention provides a rationale for predicting the efficacy of antiviral treatment of patients showing symptoms of HCV infection and/or of patients having been diagnosed with HCV infection. This rationale involves the simultaneous and/or subsequent testing of different parameters in samples of the patients and introduces the principle of the ‘early stopping rule’. It is envisaged that the kits of the present invention comprise reagents suitable for carrying out one or more of the testing steps provided in this rationale, but that the use of the kits of the present invention is not limited to testing as envisaged in this rationale. It is moreover envisaged that the kits of the present invention can comprise a paper insert describing one or more of the steps of the rationale for applying the “early stopping rule” described herein and/or the steps involved in the one ore more detection methods described herein.

The present invention provides methods for predicting the likelihood of efficacy or outcome of HCV therapy in an animal, more particularly in a human, which has been diagnosed with HCV and more particularly for a subset of patients predicting with accuracy the potential benefit of antiviral treatment. The methods of the invention involve a number of steps. Most particularly, the methods of the invention comprise the step of determining the presence of antibodies specifically directed to the HCV antigens NS4a and/or NS5a in a sample. This step of the method itself comprises a number of steps related to the actual method and reagents used for this determination step such as but not limited to and not necessarily including preparation of the sample, incubation steps, washing steps, a (qualitative or quantitative) detection step etc . . . These different steps related to the protocol of the type of determination method used (e.g. ELISA, RIA, etc.) are known to the skilled person.

The methods of the present invention relate to the determination of antibodies to HCV antigenic peptides in a sample. The sample in the context of the present invention is preferably serum or plasma. It is however envisaged that samples of other human tissues or fluids can also be used in the methods of the present invention.

The present invention is now illustrated with the following examples without being limited thereto.

### EXAMPLES

### Example 1

### General Methodology

### Example 1.1

Patient Selection and Sample Collection

Over a period of seven years detailed clinical data and specimens from chronic HCV patients participating in three therapeutic trials were collected. Study A involved untreated chronic HCV patients that were randomized to be treated with interferon-alpha (IFN-alpha mono-therapy (3×3 MU/wk, 18 months)), or with combination therapy (IFN-alpha (3×3 MU/week)+ribavirin (1000-1200 mg/day)) for 6 or 18 months. Study B involved chronic HCV patients that relapsed following IFN-alpha mono-therapy and that were retreated with a combination therapy of IFN-alpha (3×3 MU/week) and ribavirin (1000 or 1200 mg/day). The total treatment duration was 6 or 12 months. Study C was designed to study the influence of daily induction dosing (5 MU/day) versus thrice weekly administration (3×5 MU/week) of IFN-alpha mono-therapy during 4 weeks, followed by a combination with ribavirin (1000 or 1200 mg/day) during the 4 next weeks, followed by maintenance treatment with the combination IFN-alpha (3×3 MU/week)+ribavirin (1000 or 1200 mg/day) in naive chronic hepatitis C patients. The total treatment duration was 1 year. The results of the latter study have been reported before [ ]. The genotype of all infecting HCV isolates has been determined. Serum and plasma were stored at −80° C. Patient data from the different clinical trials were pooled and therapy outcome was analysed. Based on the biochemical (ALT) and virological (HCV-RNA) response of each patient, different therapeutic outcomes could be observed:1) sustained responders (SR) 6 months after therapy; 2) relapsers after therapy (REL); 3) breakthrough during therapy; 4) non-responders after therapy (NR). For the present invention, a first patient subset was created by selecting 63 SR and 62 NR from the three therapeutic trials (A, B and C). Only patients who received combination therapy have been included in this cohort. A second population consisted of 40 SR, 40 REL and 40 NR selected from trial C. Since genotype 1 is most abundant in the European region, genotype 1 positive chronic HCV-patients were selected. The 63 SR in population 1 consisted of 45 subjects with genotype Ib and 12 with genotype Ia. In the NR group (n=62), 44 were genotype Ib positive and 10 were genotype Ia positive. The other individuals had mixed genotypes la+Ib. In population 2, the 40 SR, 40 REL and 40 NR, consisted of 28, 28 and 26 HCV Ib positive patients, 8, 7 and 8 HCV Ia positive patients, and 4, 5 and 6 patients with a mixed HCV genotype 1, respectively. All serum-samples for serological testing used in the present study were obtained at the start of treatment (baseline). Samples examined for HCV-RNA content were collected at different time-points during and after treatment. All subjects gave written, informed consent to participate in these studies, which were approved by the local Ethical Review Boards. The studies were performed in accordance with the Declaration of Helsinki and its amendments taking into account Good Clinical Practice.

### Example 1.2

### HCV-RNA Determination

Measurements of serum HCV-RNA were performed with two qualitative and two quantitative representative detection methods in the context of a comparative evaluation of these methods. HCV-RNA was detected qualitatively by the Cobas Amplicor HCV 2.0 assay (Roche Diagnostics, Mannheim, Germany) and by a transcription-mediated amplification (TMA)-based assay (Versant™ HCV-RNA Qualitative Assay (Bayer Diagnostics, Emeryville, USA). The lower detection limit of the Cobas Amplicor is 50-100 IU/ml while the analytical sensitivity of the TMA-assay is 5 to 10 IU/ml [Lee et al. (2002) J Clin Microbiol 38, 4171-4179; Nolte et al. (2001) J Clin Microbiol 39, 4005-4012]. Quantitative HCV-RNA measurements were performed with the Cobas Amplicor HCV Monitor 2.0 assay (Roche Diagnostics) and with the third generation b-DNA assay (Versant™ HCV-RNA 3.0 Assay, Bayer Diagnostics). The lower detection limits of both tests are 600 IU/ml and 615 IU/ml [Beld et al. (2002) J Clin Microbiol 40, 788-793], respectively. Quantitative HCV-RNA measurements that scored ‘undetectable’ (<600 or 615 IU/ml) at the end or after therapy were retested in the TMA-assay leading to an unambiguous classification of patients as SR, REL or NR.

In population 2, the HCV-RNA decline was determined at weeks 1 and 4 of therapy. Stored serum samples (−80° C.) collected at baseline and on weeks 1 and 4 during therapy, were tested retrospectively by the quantitative Cobas Amplicor HCV Monitor 2.0 assay (Roche Diagnostics). Serum samples scoring below the detection limit of the assay (600 IU/ml) were retested using a qualitative TMA-based HCV-RNA assay (Bayer Diagnostics). The HCV-RNA log-drops at week 1 and week 4 of treatment were calculated. Samples that scored negative in the HCV Monitor assay (Roche Diagnostics) and positive in the TMA-assay, were arbitrarily given a log₁₀ value of 2.79 (=600 IU/ml). Samples scoring negative in both assays were given a log₁₀ value of 1.00.

### Example 1.3

### HCV-Genotyping

Genotyping was performed using a line-probe assay (HCV Genotype Assay (LiPA, Versant®, Bayer Diagnostics) in accordance with the manufacturer's instructions.

### Example 1.4

### Antigens

Because most of the anti-NS3 antibody produced by chronically infected humans is directed against the helicase domain, antibody reactivity to NS3 was assessed using the recombinant, E. coli-expressed, NS3 helicase domain of genotype Ib (obtained from Innogenetics N.V. (Zwijnaarde, Belgium, amino acid (M) 1188-1463) or purchased from Mikrogen GmbH (Martinsried, Germany, AA 1207-1488). Both batches were used as solid-phase ligands in EIA. Bridging experiments between both NS3-batches showed identical results. To detect antibodies according to a particular embodiment of the invention, antibodies to NS4a genotype Ia were detected using two overlapping synthetic peptides (NS4-Ia; AA1688-1707; LSGKPAIIPDREVLYREFDE [SEQ ID NO:1]) and NS4-IIa (AA17001694-1713; VLYREFDEMEECSQHLPYIE [SEQ ID NO: 2]) and in the NS5a-specific EIA two overlapping synthetic peptides NS5-Ia AA2263-2282; EDEREISVPAEILRKSRRFA [SEQ ID NO: 4]) and NS5-IIa (AA2275-2294; LRKSRRFAQALPVWARPDYN [SEQ ID NO: 5]) were used. The overlapping portion between peptides NS4-Ia and NS4-IIa is VLYREFDE [SEQ ID NO: 3] The overlapping portion between NS5-Ia and NS5-IIa is LRKSRRFA [SEQ ID NO: 6].

To detect antibodies according to a particular embodiment of the invention, antibodies to NS4a genotype Ib were detected using two overlapping synthetic peptides (NS4-Ib; M1688-1707; LSGRPAVIPDREVLYQEFDE [SEQ ID NO: 7] and NS4-IIb (NS4-IIb AA1700-1719; LYQEFDEMEECASHLPYIE [SEQ ID NO: 8] and in the NS5a-specific EIA two overlapping synthetic peptides NS5-Ib (AA2263-2282; EDEREISVPAEILRKPRKFP [SEQ ID NO: 10] and NS5-IIb (NS5-IIa AA2275-2294; LRKPRKFPPALPIWARPDYN [SEQ ID NO: II]) were used. The overlapping portion between peptides NS4-Ib and NS4-IIb is VLYQEFDE [SEQ ID NO: 9] The overlapping portion between NS5-Ib and NS5-IIb is LRKPRKFP [SEQ ID NO: 12]. All peptides were synthesized with a multiple peptide synthesizer using standard Fmoc chemistry (Syro, MultiSyn Tech). Peptides were purified by high-performance liquid chromatography using standard protocols.

### Example 1.5

### Enzyme Immunoassays (EIAs) for the Detection of Antibodies to NS3, NS4a and NS5a

All EIAs were essentially performed as described (23). Recombinant proteins (rNS3) or peptides (NS4a and NS5a) were passively adsorbed at 4° C. to 96-well microtiter plates (Maxisorb, Nunc, Copenhagen, Denmark) at 1 μg/ml (proteins) or 10 μg/ml (peptides) in 50 mM sodium carbonate buffer (pH 9,6). The plates were then blocked by incubation with dilution buffer containing PBS, 2% goat serum, and 1% bovine serum albumin for two hours at 37° C. Serial dilutions of sera, starting at 1:50 or 1:10 or undiluted, were then added to the plates for 90 minutes. Bound serum antibodies were detected by a peroxidase-conjugated rabbit anti-human IgG (DAKO) followed by addition of the substrate TMB (Sigma T-8665). The reaction was stopped by addition of 1M NaOH and absorbencies were subsequently read at 450 nm. The EIA cut-off value was calculated by adding 3 times the standard deviation to the mean value of 3 serially diluted anti-HCV-negative sera used in each assay. Results have been expressed as end-point titers. Since all antigens and peptides in the EIA are of genotype 1 and since only tested genotype 1-infected individuals were tested, there was a correct genotype match for these antibody determinations.

### Example 1.6

### HCV Core Antigen Determination

Antibodies against HCV core antigen have been described for example in. Orito et al. Gut (1996) 39:876-880 and in Aoyagi K, J Clin Microbiol 1999;37:18021808. Such antibodies allow the quantitative determination of HCV core antigen. A standardized commercial assay has been developed for the quantitative detection of HCV core Ag (Ortho-Clinical Diagnostics, Raritan, NJ). Herein, 100 microliter of samples and controls are mixed with 50 microliter of a pretreatment buffer containing detergents and pretreated for 30 minutes at 56° C. to dissociate HCV core antigen-antibody complexes. For the ELISA reaction, 100 microliter of pretreated samples and controls are incubated for 60 minutes at 25° C. in monoclonal antibody-coated wells of a microtiter plate. The plates are washed and incubated for 30 minutes at 25° C. with 200 microliter of conjugate, washed again, and incubated for 30 minutes at 25° C. with 200 microliter of substrate. The optical densities (ODs) are read in a spectrophotometer at 490 nm using a 620 nm reference. The samples and controls are tested in duplicate and the mean OD of each duplicate testing is used. The samples that exhibit more than 25% variation between the two ODs are considered invalid and retested. The lower detection cutoff is established for each run and corresponds to the mean OD of the 2 negative controls plus 0.041. A sample is considered positive only when the mean OD is higher than the cutoff OD of the corresponding run. The amount of total HCV core Ag in pg/mL is calculated by means of a standard curve established in each run by testing serial dilutions of a standard containing 400 HCV core Ag pg/mL.

### Example 1.7

### Statistical Analyses

X² test was used for comparison of frequencies between different groups. Relative Risk (RR) values are calculated by Woolfs method. To compare the quantity of antibodies between different groups, the Mann-Whitney U test was used. Data are presented in a box-and-whisker plot, wherein the lower and upper limits of the central box represent the 25^{th} and 75^{th} percentile. The middle line represents the median value. A line extends from the minimum to the maximum value, excluding ‘outside’ and ‘far out’ values which are displayed as separate points.

Negative predictive value (NPV, %) refers to the true negatives/true+false negatives, and PPV (positive predictive value, %) refers to the true positives/true+false positives. In case of one parameter (e.g. anti-NS4a), the NPV-value for SR (anti-NS4a >1) equals the PPV-value for NR (anti-NS4a <1). In case of two parameters, however, care must be taken and both values are not always interchangeable.

### Example 2

### Occurrence of Antibodies to HCV-Specific Non-Structural Proteins in Chronic HCV Patients

In a first series of experiments anti-NS3, anti-NS4a and anti-NS5a antibodies were determined in chronic HCV-patients at the onset of therapy (week 0), when HCV-RNA was still positive. Antibodies to NS3 were only measured in population 1 and were present in all patients, irrespective of therapy outcome (SR vs NR, p>0,05, not significant) (table 1).

TABLE 1
Frequency of antibodies to non-structural (NS) proteins
of HCV in 2 populations of chronic HCV patients.

| | | SR | REL | NR | | | PPV | NPV |
|---|---|---|---|---|---|---|---|---|
| | | #/total | #/total | #/total | RR | P-value | for SR | for SR |
| | | (%) | (%) | (%) | for SR | (X²-test) | % | % |
| Pop. 1 | anti-NS4a >1 | 54⁺/62 | | 30⁺/62 | 7.2 | 4.0 10⁻⁶ | 64.3 | 80 |
| | | (87) | | (48) | | | | |
| | anti-NS5a >1 | 49⁺/60 | | 24⁺/54 | 5.6 | 3.5 10⁻⁵ | 67.1 | 73.2 |
| | | (82) | | (44) | | | | |
| Pop. 2 | anti-NS4a >1 | 36⁺/40 | 29⁺/38 | 21⁺/39 | 4.9 | 3.0 10⁻³ | 41.9 | 87.5 |
| | | (90) | (76) | (54) | | | | |
| | anti-NS5a >1 | 37⁺/40 | 32⁺/38 | 17⁺/31 | 5.0 | 8.0 10⁻³ | 43 | 87 |
| | | (93) | (84) | (55) | | | | |

Antibodies to NS4a and NS5a were determined at the onset of therapy (week 0), when HCV-RNA was still positive in all participating individuals. Anti-NS4a was measured in 62 SR (sustained responders) and 62 NR (non-responders) (population 1), and in 40 SR, 38 REL (relapsers) and 39 NR (population 2), respectively. Anti-NS5a was measured in 60 SR and 54 NR (population 1) and in 40 SR, 38 REL and 31 NR (population 2), respectively.‘*’ refers to the number of individuals that score positive for the analysed antibody. Relative Risk (RR) values are calculated by Woolf's method, and the significance of it's deviation from unity is estimated by X² test. The RR indicates how many times more frequent the SR are positive for the antibody tested compared to the NSR (non-sustained responders =REL+NR).

As shown in Table 1, anti-NS4a and -NS5a occurred more frequently in SR than in NR (RR=7,2 and p=4,0 10-6 for anti-NS4a; RR=5,6 and p=3,5 10⁻⁵ for anti-NS5a). Indeed, 87% of the SR and only 48% of the NR had antibodies to NS4a at the start of therapy. Anti-NS5a antibodies were present in 82% of SR and in 44% of NR. Since population I consisted of SR on the one hand and NR on the other hand, the occurrence of these antibodies in a second population (population 2) were retested wherein also the relapsers to therapy (REL), were included. In this population, SR were compared to NSR (non-sustained responders=REL+NR), and anti-NS4a and -NS5a antibodies also occurred more frequently in SR than in NSR, albeit with a lower significance. Table 2 summarizes the results of these analyses at different cut-off levels of the EIA. This was done to examine whether a higher EIA cut-off level for both antibodies showed a stronger correlation with therapy outcome.

TABLE 2
Negative Predictive Value (NPV) of the presence of anti-NS4a or anti-NS5a
antibodies for the sustained response to combination therapy.

| | | SR | REL | NR | | | NPV |
|---|---|---|---|---|---|---|---|
| | | #/total | #/total | #/total | RR | P-value | for SR |
| | | (%) | (%) | (%) | for SR | (X²-test) | % |
| Pop. 1 | anti-NS4a >1 | 54⁺/62 | | 30⁺/62 | 7.2 | 4.0 10⁻⁶ | 80 |
| | | (87) | | (48) | | | |
| | anti-NS4a >10 | 51⁺/62 | | 26⁺/62 | 6.4 | 3.7 10⁻⁶ | 76.1 |
| | | (82) | | (42) | | | |
| | anti-NS4a >50 | 50⁺/62 | | 21⁺/62 | 8.1 | 1.4 10⁻⁷ | 77.4 |
| | | (81) | | (34) | | | |
| | anti-NS4a >100 | 48⁺/62 | | 19⁺/62 | 7.8 | 1.7 10⁻⁷ | 75.4 |
| | | (77) | | (31) | | | |
| | anti-NS5a >1 | 49⁺/60 | | 24⁺/54 | 5.6 | 3.5 10⁻⁵ | 73.2 |
| | | (82) | | (44) | | | |
| | anti-NS5a >10 | 46⁺/60 | | 19⁺/54 | 6.1 | 7.9 10⁻⁶ | 71.4 |
| | | (77) | | (35) | | | |
| | anti-NS5a >50 | 42⁺/60 | | 17⁺/54 | 5.1 | 4.0 10⁻⁵ | 67.3 |
| | | (70) | | (32) | | | |
| | anti-NS5a >100 | 40⁺/60 | | 14⁺/54 | 5.7 | 1.4 10⁻⁵ | 66.7 |
| | | (67) | | (26) | | | |
| Pop. 2 | anti-NS4a >1 | 36⁺/40 | 29⁺/38 | 21⁺/39 | 4.9 | 0.003 | 87.5 |
| | | (90) | (76) | (54) | | | |
| | anti-NS4a >10 | 33⁺/40 | 28⁺/38 | 19⁺/39 | 3.0 | 0.018 | 81.1 |
| | | (83) | (74) | (49) | | | |
| | anti-NS4a >50 | 32⁺/40 | 25⁺/38 | 14⁺/39 | 3.9 | 0.002 | 82.6 |
| | | (80) | (66) | (36) | | | |
| | anti-NS4a >100 | 30⁺/40 | 24⁺/38 | 12⁺/39 | 3.4 | 0.003 | 80.4 |
| | | (75) | (63) | (31) | | | |
| | anti-NS5a >1 | 37⁺/40 | 32⁺/38 | 17⁺/31 | 5.0 | 0.008 | 87 |
| | | (93) | (84) | (55) | | | |
| | anti-NS5a >10 | 34⁺/40 | 29⁺/38 | 13⁺/31 | 3.6 | 0.008 | 81.8 |
| | | (85) | (76) | (42) | | | |
| | anti-NS5a >50 | 30⁺/40 | 25⁺/38 | 12⁺/31 | 2.6 | 0.03 | 76.2 |
| | | (75) | (66) | (39) | | | |
| | anti-NS5a >100 | 28⁺/40 | 22⁺/38 | 9⁺/31 | 2.9 | 0.011 | 76 |
| | | (70) | (58) | (29) | | | |

Relative Risk (RR) values are calculated by Woolfs method, and the significance of it's deviation from unity is estimated by X² test. The RR indicates how many times more frequent the SR are positive for the antibodies tested compared to the NSR (non-sustained responders =REL+NR). The NPV (=true negatives/true+false negatives) predicts that 80% of anti-NS4a negative patients (=1, Pop. 1) will be NR (non-responders) after treatment. NPV for SR was considered at different EIA cut-off levels for anti-NS4a and anti-NS5a.

The data in table 2 demonstrate that the results remain significant until a cut-off of at least 1/100 end-point dilution was applied. To analyse whether these data have any predictive value in determining NSR to therapy, the negative predictive value (NPV) of the presence of these antibodies for SR to antiviral therapy were calculated. The presence of anti-NS4a alone at cut-off level 1 has a NPV for SR of 80% and 87,5% in population 1 and 2, respectively. Increasing the cut-off level does not improve the NPV for SR. For anti-NS5a antibodies, the NPV for SR at cut-off level I are 73,2% and 87% for population 1 and 2, respectively. Increasing the cut-off level for anti-NS5a, does not increase the NPV. In conclusion, the presence (cut-off level 1) of anti-NS4a and anti-NS5a is significantly associated with SR, or, inversely their absence is associated with NSR.

### Example 3

### Magnitude of Antibody Responses to NS3, NS4a and NS5a Proteins in Chronic HCV Patients

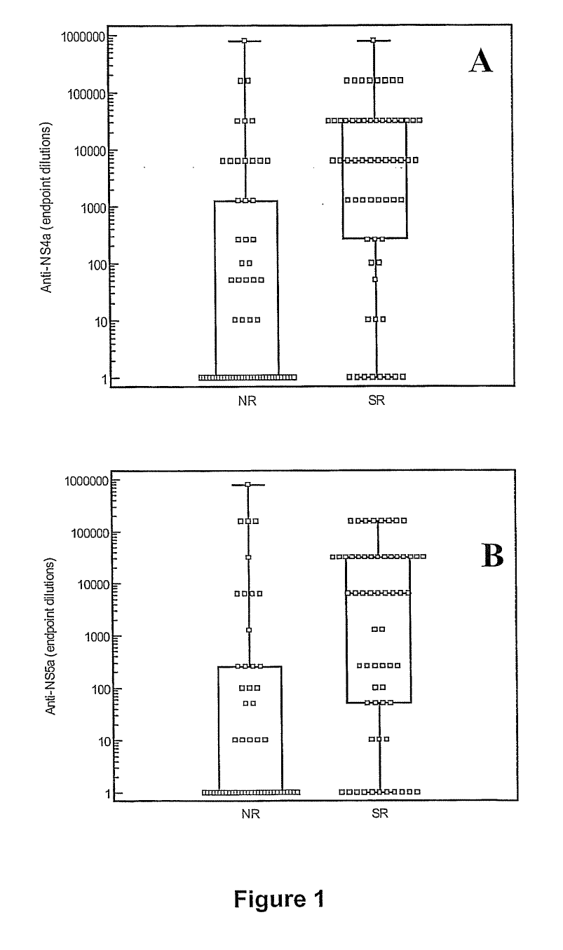

The magnitude of the antibody responses to NS3, NS4a and NS5a was compared between SR and NR in population 1 (FIG. 1). The anti-NS3 antibody levels of SR and NR did not differ significantly. However, patients displaying a SR to therapy had significantly higher levels of antibodies to NS4a and NS5a than NR (p=1,2 10-7 for anti-NS4a and p=5,1 10⁻⁶ for anti-NS5a).

Comparisons made in population 2 (results not shown) revealed that the levels of anti-NS4a and anti-NS5a differed significantly between SR, REL and NR, respectively (anti-NS4a: SR vs REL (p<0,01); REL vs NR (p-0,01); SR vs NR (p<0,0001) and anti-NS5a: SR vs REL (p<0,01); REL vs NR (p=0,05); SR vs NR (p<0,0005)).

### Example 4

### Simultaneous Absence of Antibodies to HCV-Specific Non-Structural Proteins in Chronic HCV Patients

Firstly, it was examined whether the simultaneous absence of anti-NS4a 15 and anti-NS5a was stronger correlated with a negative therapy outcome than the absence of each antibody alone. To do so the positive predictive value (PPV) was calculated for NSR to antiviral therapy of the simultaneous absence of anti-NS4a and anti-NS5a antibodies (Table 3).

TABLE 3
Positive Predictive Value (PPV) of the simultaneous absence of antibodies
to NS4a and NS5a for the non-sustained response to combination therapy.

| | | | NSR | | | | |
|---|---|---|---|---|---|---|---|
| | | SR | REL | NR | | | PPV |
| | | #/total | #/total | #/total | RR | P-value | for NSR |
| | | (%) | (%) | (%) | for NSR | (X²-test) | % |
| Pop. 1 | anti-NS4a = 1 and | 5/59 | | 20/54 | 6.3 | 2.6 10⁻⁴ | 80.0 |
| | anti-NS5a = 1 | (8) | | (37) | | | |
| | anti-NS4a ≦10 and | 7/59 | | 25/54 | 6.4 | 5.0 10⁻⁵ | 78.1 |
| | anti-NS5a ≦10 | (12) | | (46) | | | |
| | anti-NS4a ≦50 and | 7/59 | | 29/54 | 8.6 | 1.8 10⁻⁶ | 80.6 |
| | anti-NS5a ≦50 | (12) | | (54) | | | |
| | anti-NS4a ≦100 and | 7/59 | | 31/54 | 10.0 | 3.1 10⁻⁷ | 81.6 |
| | anti-NS5a ≦100 | (12) | | (57) | | | |
| Pop. 2 | anti-NS4a = 1 and | 2/40 | 2/38 | 8/31 | 3.2 | 0.13 | 83.3 |
| | anti-NS5a = 1 | (5) | (5) | (26) | | | |
| | anti-NS4a ≦10 and | 4/40 | 2/38 | 12/31 | 2.3 | 0.16 | 77.8 |
| | anti-NS5a ≦10 | (10) | (5) | (39) | | | |
| | anti-NS4a ≦50 and | 4/40 | 6/38 | 15/31 | 3.9 | 0.014 | 84.0 |
| | anti-NS5a ≦50 | (10) | (16) | (48) | | | |
| | anti-NS4a ≦100 and | 4/40 | 7/38 | 17/31 | 4.8 | 0.004 | 85.7 |
| | anti-NS5a ≦100 | (10) | (18) | (55) | | | |

Antibodies were determined at the onset of therapy (week 0), when HCV-RNA was still positive in all participating individuals. The simultaneous absence of anti-NS4a and anti-NS5a was analysed. Double negative patients lack both antibodies. Relative Risk (RR) values are calculated by Woolfs method, and the significance of it's deviation from unity is estimated by X² test. The PPV predicts that 80% of anti-NS4a negative (=1) and anti-NS5a negative (=1) patients (Pop. 1) will be non-sustained responder (NSR) after treatment. PPV for NR was considered at different EIA cut-off levels for anti-NS4a and anti-NS5a.

As shown in Table 2, the absence of anti-NS4a alone and anti-NS5a alone at an EIA cut-off level of 1 has a PPV for NR to therapy of 80-87,5% and 73,2-87%, respectively. The simultaneous absence of both antibodies has a PPV of 80-83,3%, meaning that individuals lacking both anti-NS4a and anti-NS5a at baseline have 80-83,3% ‘chance’ of being NSR to treatment. The simultaneous absence of both antibodies does not remarkably improve the PPV for NSR compared to the absence of each antibody alone.

### Example 5

### Quantitative HCV-RNA Measurement and the Absence of Anti-NS4a or Anti-NS5a Antibodies for Predicting Non-Sustained Response to Therapy in Chronic HCV Patients

Minimizing the loss of potential responders is the most important clinical goal in defining an early stopping rule. It is preferable to maximize the PPV for NSR or to minimize the discontinuation of treatment in patients who might ultimately respond after completion of the full course of therapy. Therefore for this particular purpose, a PPV for NSR of 87% is not high enough and it was sought to improve the predictive value for NR by combining the absence of antibodies against NS4a or NS5a with different viral load measurements. Firstly, the absence of antibodies was combined with pre-treatment HCV-RNA levels. These combinations, however, did not increase the predictive value for NSR. In a second series of analyses it was examined whether the absence of a 2 log₁₀ HCV-RNA decline at treatment week 1 or 4, combined with the baseline absence of anti-NS4a or -NS5a, increased the PPV for NSR. Table 4 demonstrates that the absence of a 2 log viral decline at weeks 1 and 4 has a predictive value for NSR of 74,7 and 85,2%, respectively. Combining these data with the antibody data did not enhance the predictive value.

However, combining HCV-RNA measurements at week 1 of therapy with the absence of antibodies at baseline maximizes the predictive value (Table 4).

TABLE 4
Factors for predicting non-sustained response
after IFNalpha/ribavirin therapy.

| | PPV | sensitivity | specificity |
|---|---|---|---|
| Pop. 2 | for NSR | (%) | (%) |
| HCV-RNA >5 log₁₀ IU/ml (W1) | 87.7 | 68 | 82 |
| no 2 log viral decline (W1) | 74.7 | 90 | 44 |
| no 2 log viral decline (W4) | 85.2 | 69 | 77 |
| anti-NS4a = 1 (W0) | 87.1 | 35 | 90 |
| anti-NS4a = 1 (W0) and | 100 | 28 | 100 |
| HCV-RNA >5 log₁₀ IU/ml (W1) | | | |
| anti-NS4a ≦10 (W0) and | 100 | 29 | 100 |
| HCV-RNA >5 log₁₀ IU/ml (W1) | | | |
| anti-NS4a ≦50 (W0) and | 100 | 39 | 100 |
| HCV-RNA >5 log₁₀ IU/ml (W1) | | | |
| anti-NS4a ≦100 (W0) and | 96.9 | 43 | 97 |
| HCV-RNA >5 log₁₀ IU/ml (W1) | | | |
| anti-NS5a = 1 (W0) | 87.0 | 29 | 93 |
| anti-NS5a = 1 (W0) and | 100 | 22 | 100 |
| HCV-RNA >5 log₁₀ IU/ml (W1) | | | |
| anti-NS5a ≦10 (W0) and | 95.0 | 30 | 97 |
| HCV-RNA >5 log₁₀ IU/ml (W1) | | | |
| anti-NS5a ≦50 (W0) and | 95.5 | 33 | 97 |
| HCV-RNA >5 log₁₀ IU/ml (W1) | | | |
| anti-NS5a ≦100 (W0) and | 96.0 | 38 | 97 |
| HCV-RNA >5 log₁₀ IU/ml (W1) | | | |

Sensitivity here describes the proportion (%) of NSR patients having the parameter, whereas specificity describes the proportion (%) of SR patients not having the parameter.

A viral load at treatment week 1 exceeding 100 000 IU/ml (=5 log₁₀) has a predictive value for NSR of 87,7%. Combining this decision limit with the absence of anti-NS4a or anti-NS5a at baseline results in a PPV for NSR of 100%. For anti-NS4a, cut-off levels up to <50 EIA end-point dilutions result in a 100% predictive value. For anti-NS5a, only the absence (cut-off level =1) results in 100% prediction. Therefore, the absence of anti-NS4a (or anti-NS5a) at baseline combined with a HCV-RNA level >100 000 IU/ml at week 1 of therapy predicts NSR with 100% certainty. Using this combination of test results as a stopping rule will deprive no patient from a possible beneficial treatment. Sensitivity here describes the proportion of NSR patients fulfilling the threshold (e.g. anti-NS4a =1) from all NSR patients, specificity describes the proportion of SR patients not fulfilling the threshold from all SR patients. At a threshold of anti-NS4a of <50 and HCV RNA at week 1 of 105, 40% of future NSR are detected while no SR will be excluded. Since the rule was designed to minimize discontinuation of treatment in patients who might ultimately respond, one has to accept this low sensitivity.

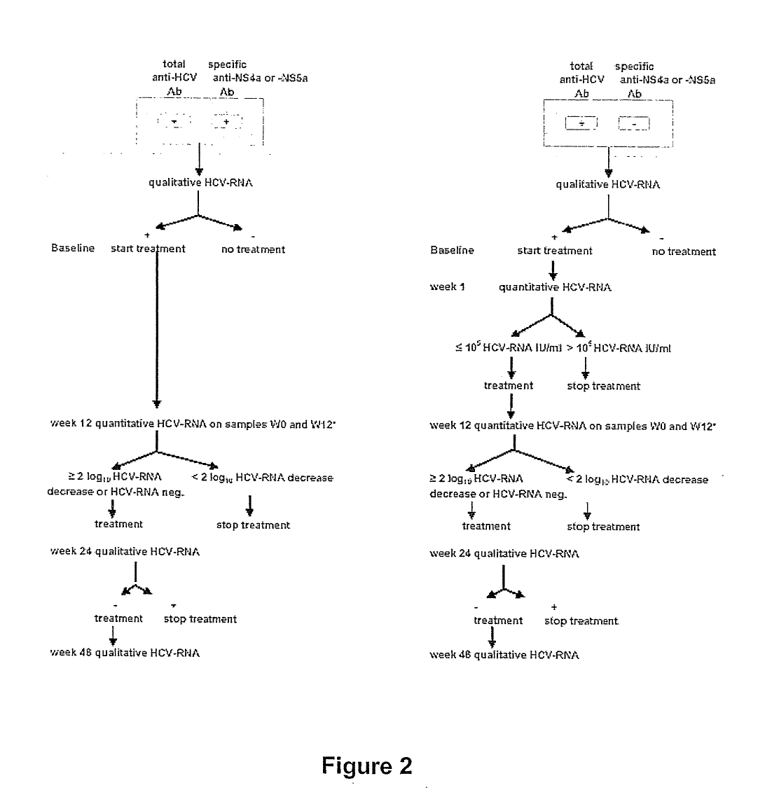

Based on the previous findings an algorithm for the management of antiviral therapy in patients with chronic HCV infection is proposed in accordance with a particular embodiment of the invention and shown in FIG. 2. It is suggested that all patients are tested for anti-NS4a or -NS5a at baseline. This test can be done in combination with the usual anti-HCV Ab screening test. In patients positive for anti-HCV antibodies and HCV-RNA but negative for anti-NS4a and/or -NS5a, it is proposed to start treatment and to quantify the HCV-RNA after 1 week of therapy. In patients with a viral load above 100 000 IU/ml after 1 week, treatment can be discontinued. In patients with HCV-RNA levels lower than 100 000 IU/ml, therapy is preferably continued until week 12. From week 12 on, the recommendations made during previous consensus conferences can be followed.

### Example 6

### HCV-Core Antigen Measurement and anti-NS4a or anti-NS5a Antibodies for Predicting Non-Sustained Response to Therapy in Chronic HCV Patients.

Example 5 illustrates one embodiment of the invention wherein the success of an interferon-based antiviral HCV treatment determined by determining antibody concentrations against NS4a and NS5a, in combination with the quantitative determination of the HCV concentration. This quantitative determination is generally performed by assaying the concentration of HCV RNA. Assaying a compound of human or viral origin which concentration is tightly correlated (regression coefficient being at least 0,80, at least 0,85 or at least 0,90) with the concentration of HCV RNA would equally suit for performing the present invention. Such compound which concentration is being tightly linked (r=0.92) with the HCV RNA concentration is the HCV core antigen as described in Bouvier-Alias et al (2002) Hepatology 36, 211-218. The HCV-core antigen (Ag) quantification was assessed using the Ortho trak-C assay (Ortho-Clinical Diagnostics, Raritan, NJ; kit lot number DCAO12) according to Bouvier Alias et al. [cited above].

In the dataset describe previously, the concentration of core antigen as an alternative to RNA determination was determined. A HCV-RNA level of 10⁵ lU/ml was found to correlate with a HCV-core Ag level of 15 pg/ml (y=0,0002, x=0,3407; R²=0,87). When applying a threshold of 15 pg/ml, the HCV-core Ag test has a diagnostic sensitivity for NSR at week 1 of 83% (Table 5).

TABLE 5
Factors for predicting non-sustained response (NSR) at baseline and/or
after 1, 4 or 12 weeks of IFNα/ribavirin combination therapy.

| | | NSR | | | | |
|---|---|---|---|---|---|---|
| | | | | PPV | | |
| | SR | REL | NR | for NSR | Sensitivity | Specificity |
| | #/total | #/total | #/total | % | % | % |
| HCV-RNA >5 log10 | 7/39 | 15/35 | 35/39 | 87.7 | 67.6 | 82.1 |
| IU/ml (week 1) | | | | | | |
| HCV-core Ag >15 | 9/40 | 11/36 | 34/39 | 83.3 | 60.0 | 77.5 |
| pg/ml (week 1) | | | | | | |
| no 2 log viral decline* | 22/39 | 28/35 | 37/37 | 74.7 | 90.3 | 43.6 |
| (week 1) | | | | | | |
| no 2 log viral decline* | 9/39 | 15/37 | 37/38 | 85.2 | 69.3 | 76.9 |
| (week 4) | | | | | | |
| no 2 log viral decline* | 0/40 | 2/40 | 40/40 | 100 | 52.5 | 100 |
| (week 12) | | | | | | |
| anti-NS4a = 1 | 0/39 | 4/34 | 16/38 | 100 | 27.8 | 100 |
| (baseline) and HCV- | | | | | | |
| RNA >5 log10 IU/ml | | | | | | |
| (week 1) | | | | | | |
| anti-NS4a = 1 | 0/40 | 1/35 | 16/38 | 100 | 23.3 | 100 |
| (baseline) and HCV- | | | | | | |
| core Ag >15 pg/ml | | | | | | |
| (week 1) | | | | | | |
| anti-NS4a = 10 | 0/39 | 4/34 | 17/38 | 100 | 29.2 | 100 |
| (baseline) and HCV- | | | | | | |
| RNA >5 log10 IU/ml | | | | | | |
| (week 1) | | | | | | |
| anti-NS4a = 10 | 0/40 | 2/35 | 18/38 | 100 | 27.4 | 100 |
| (baseline) and HCV- | | | | | | |
| core Ag >15 pg/ml | | | | | | |
| (week 1) | | | | | | |
| anti-NS4a = 50 | 0/39 | 6/34 | 22/38 | 100 | 38.9 | 100 |
| (baseline) and HCV- | | | | | | |
| RNA >5 log10 IU/ml | | | | | | |
| (week 1) | | | | | | |
| anti-NS4a = 50 | 0/40 | 4/35 | 23/38 | 100 | 37.0 | 100 |
| (baseline) and HCV- | | | | | | |
| core Ag >15 pg/ml | | | | | | |
| (week 1) | | | | | | |
| anti-NS4a = 100 | 1/39 | 7/34 | 24/38 | 96.9 | 43.1 | 97.4 |
| (baseline) and HCV- | | | | | | |
| RNA >5 log10 IU/ml | | | | | | |
| (week 1) | | | | | | |
| anti-NS4a = 100 | 1/40 | 4/35 | 23/38 | 96.4 | 37.0 | 97.5 |
| (baseline) and HCV- | | | | | | |
| core Ag >15 pg/ml | | | | | | |
| (week 1) | | | | | | |
| anti-NS5a = 1 | 0/39 | 2/34 | 12/30 | 100 | 21.9 | 100 |
| (baseline) and HCV- | | | | | | |
| RNA >5 log10 IU/ml | | | | | | |
| (week 1) | | | | | | |
| anti-NS5a = 1 | 0/40 | 2/35 | 13/30 | 100 | 23.1 | 100 |
| (baseline) and HCV- | | | | | | |
| core Ag >15 pg/ml | | | | | | |
| (week 1) | | | | | | |
| anti-NS5a = 10 | 1/39 | 3/34 | 16/30 | 95.0 | 29.7 | 97.4 |
| (baseline) and HCV- | | | | | | |
| RNA >5 log10 IU/ml | | | | | | |
| (week 1) | | | | | | |
| anti-NS5a = 10 | 1/40 | 2/35 | 17/30 | 95.0 | 29.2 | 97.5 |
| (baseline) and HCV- | | | | | | |
| core Ag >15 pg/ml | | | | | | |
| (week 1) | | | | | | |
| anti-NS5a = 50 | 1/39 | 4/34 | 17/30 | 95.5 | 32.8 | 97.4 |
| (baseline) and HCV- | | | | | | |
| RNA >5 log10 IU/ml | | | | | | |
| (week 1) | | | | | | |
| anti-NS5a = 50 | 1/40 | 4/35 | 18/30 | 95.7 | 33.8 | 97.5 |
| (baseline) and HCV- | | | | | | |
| core Ag >15 pg/ml | | | | | | |
| (week 1) | | | | | | |
| anti-NS5a = 100 | 1/39 | 5/34 | 19/30 | 96.0 | 37.5 | 97.4 |
| (baseline) and HCV- | | | | | | |
| RNA >5 log10 IU/ml | | | | | | |
| (week 1) | | | | | | |
| anti-NS5a = 100 | 1/40 | 6/35 | 20/30 | 96.3 | 40.0 | 97.5 |
| (baseline) and HCV- | | | | | | |
| core Ag >15 pg/ml | | | | | | |
| (week 1) | | | | | | |

Combining this decision limit with the absence of anti-NS4a or anti-NS5a at baseline, results in a PPV for NSR of 100%. The quantification of HCV-RNA after 1 week of therapy can thus be replaced by a quantitative HCV-core Ag determination. In patients with HCV-core Ag levels >15 pg/mi, treatment can be discontinued.

### Example 7

### Relevance of the Proposed Algorithm of the Invention in peg-IFN-alpha+Ribavirin Treatment of Chronic HCV Patients

The predictive algorithm of the present invention was initially derived from data obtained in patients treated with IFN-alphalribavirin. In addition the algorithm as well in patients treated with peg-IFNalpha/ribavirin were tested. This was performed using a group of 35 chronic genotype I HCV patients that were treated for 12 months with peg-IFN-alphafribavirin (Group 2). In Table 6 these patients have been listed according to their treatment-response.

TABLE 6
Treatment outcome and anti-NS4a status in 35 chronic HCV patients
who were (re-)treated with peg-IFN-α/ribavirin.

| | | | peg-IFN-α + ribavirin treatment | | | |
|---|---|---|---|---|---|---|
| | | | | baseline | | |
| patient-ID | | | | anti-NS4a | | week 1 |
| | previous | | | (end-point | HCV-RNA | HCV-RNA |
| # | outcome | Genotype | outcome | dil.) | (IU/ml) | (IU/ml) |
| 1 | naive | 1a | SR | 156250 | 1 500 000 | 138 000 |
| 2 | naive | 1b | SR | 1 | 56 300 | 11 300 |
| 3 | naive | 1b | SR | 1250 | 144 000 | not |
| | | | | | | available |
| 4 | naive | 1b | SR | 31250 | >700 000 | 532 000 |
| 5 | naive | 1b | SR | 6250 | 327 000 | 177 000 |
| 6 | naive | 1a | SR | 6250 | >700 000 | 56 500 |
| 7 | re-treated REL | 1b | SR | 1250 | 170 000 | 28 400 |
| 8 | re-treated REL | 1b | SR | 156250 | 402 000 | 2340 |
| 9 | re-treated REL | 1b | SR | 250 | 6 868 800 | <600 |
| 10 | re-treated REL | 1b | SR | 6250 | 189 000 | 128 000 |
| 11 | re-treated REL | 1a + 1b | SR | 6250 | 324 456 | not |
| | | | | | | available |
| 12 | re-treated REL | 1b | SR | 1250 | 1 627 220 | 40 700 |
| 13 | re-treated REL | 1b | SR | 250 | 125 000 | <600 |
| 14 | re-treated REL | 1b | SR | 1250 | 9 385 000 | 3 931 050 |
| 15 | re-treated REL | 1b | SR | 31250 | 215 000 | 64 900 |
| 16 | re-treated REL | 1a | SR | 31250 | 144 000 | 11 400 |
| 17 | re-treated REL | 1b | SR | 31250 | >700 000 | >700 000 |
| 18 | re-treated REL | 1b | SR | 1 | 570 000 | 90 000 |
| 1 | re-treated NR | 1b | REL | 1 | 287 000 | 4040 |
| 2 | re-treated NR | 1b | REL | 1 | >700 000 | not |
| | | | | | | available |
| 3 | re-treated NR | 1b | REL | 1 | 454 000 | 211 000 |
| 4 | re-treated REL | 1b | REL | 1 | 522 000 | 422 000 |
| 5 | re-treated REL | 1a + 1b | REL | 250 | 303 000 | 277 000 |
| 1 | naive | 1a | NR | 1 | 4 500 000 | 2 750 000 |
| 2 | naive | 1a | NR | 1 | 3 700 000 | 1 700 000 |
| 3 | naive | 1a | NR | 1 | 615 000 | 471 000 |
| 4 | naive | 1b | NR | 6250 | 1 200 000 | 821 000 |
| 5 | naive | 1b | NR | 1 | 1 790 000 | 327 000 |
| 6 | re-treated NR | 1b | NR | 1 | >700 000 | 5 270 000 |
| 7 | re-treated NR | 1b | NR | 1 | >700 000 | 473 000 |
| 8 | re-treated NR | 1b | NR | 1 | 706 000 | 377 000 |
| 9 | re-treated NR | 1a + 1b | NR | 1 | 1 780 000 | 1 450 000 |
| 10 | re-treated NR | 1b | NR | 1250 | >700 000 | >700 000 |
| 11 | re-treated NR | 1b | NR | 1 | 5 300 000 | 310 000 |
| 12 | re-treated NR | 1a + 1b | NR | 31250 | 942 000 | 267 000 |

There were 18 SR, 5 REL and 12 NR. Only two of the 18 SR had no anti-NS4a antibodies at baseline but after one week of treatment their HCV-RNA level dropped below 105 IU/mi. In REL and NR, antibodies were absent in 4 (out of 5) and 9 (out of 12) patients, respectively, but in these 4 antibody-positive subjects no drop of HCV-RNA below 1 05 IU/mi was noted. Therefore the algorithm based on baseline anti-NS4a and HCV-RNA levels after one week is equally valid in patients treated with peg-IFN-alpha/ribavirin. Of the 14 retreated REL, 12 had anti-NS4a antibodies prior to treatment and 11 of these turned out to be SR to a treatment with peg-IFN-alphafribavirin.

As shown in table 7, the baseline absence of anti-NS4a alone has a PPV for NSR to peg-lFNalpha/ribavirin treatment of 86,7% and thus equals the previously observed predictive value.

TABLE 7
Factors for predicting non-sustained response (NSR) after 1 week of peg-IFN-α/ribavirin therapy.

| | | NSR | | | | | | |
|---|---|---|---|---|---|---|---|---|
| | SR | REL | NR | | | PPV | | |
| | #/total | #/total | #/total | RR | P-value | for NSR | Sensitivity | Specificity |
| population 3 | (%) | (%) | (%) | for NSR | (c2-test) | % | % | % |
| anti-NS4a = 10 | 2/18 | 4/5 | 9/12 | 26 | 9.4 10 − 5 | 86.7 | 76.5 | 88.9 |
| (W0) | (11) | (80) | (75) | | | | | |
| HCV-RNA >5 log10 | 6/16 | 3/4 | 12/12 | 25 | 8.0 10 − 4 | 71.4 | 93.8 | 62.5 |
| IU/ml (W1) | (38) | (75) | (100) | | | | | |
| anti-NS4a = 10 | 0/16 | 2/4 | 9/12 | 69 | 4.2 10 − 5 | 100 | 68.8 | 100 |
| (W0) and HCV-RNA >5 | (0) | (50) | (75) | | | | | |
| log10 IU/ml (W1) | | | | | | | | |

The sensitivity of the anti-NS4a marker was found markedly enhanced in this group of peg-IFN-alpha/ribavirin treated patients (77% vs 41%), probably because of a selection bias due to the inclusion of a high number of REL to a former IFN-alpha/ribavirin treatment. Finally, it was demonstrated that combining a viral load at treatment week 1 exceeding 105 IU/ml with the absence of anti-NS4a at baseline predicts NSR with 100% certainty in patients treated with peg-IFN-alpha /ribavirin.

The finding that baseline serum levels of anti-NS4a are significantly correlated with SR to therapy was confirmed in this group. Moreover, the proposed ‘stopping rule’, wherein a viral load at treatment week 1 exceeding 1 05 IU/ml is combined with the baseline absence of anti-NS4a also holds for these patients. The observed sensitivity (69%) of this combination of markers in predicting 100% NSR is far more elevated than has been published so far. This is possibly due to the presence in this cohort of a high number of REL to a former IFN-alpha/ribavirin treatment. All but one of the former REL with antibodies to NS4a became SR following peg-IFN-alpha/ribavirin therapy. This treatment appears to induce more SR in patients with anti-NS4a antibodies than the former therapy with IFN-alpha/ribavirin.

1-33. (canceled)

34. A kit comprising one or more HCV antigenic peptides, each of said one or more HCV antigenic peptides being selected from:
HCV antigenic peptides comprising in their sequence at least 8 amino acids of the NS4a protein of HCV and/or
HCV antigenic peptides comprising in their sequence at least 8 amino acids of the NS5a protein sequence of HCV.

35. The kit according to claim 34, which comprises:
(a) at least one HCV antigenic peptide comprising in its sequence at least 8 amino acids of the NS4a protein of HCV, and
(b) at least one HCV antigenic peptide comprising in its sequence at least 8 amino acids of the NS5a protein of HCV, and wherein, detection of binding of antibodies to said peptide(s) listed under (a) and said peptide(s) listed under (b) is ensured separately.

36. The kit according to claim 35, wherein said peptides listed under (a) and said peptide(s) listed under (b) are physically separated from each other.

37. The kit according to claim 34, wherein said one or more HCV antigenic peptides comprising in their sequence at least 8 amino acids of the NS4a protein sequence, are peptides selected from the group consisting of:
peptides comprising in their sequence at least 8 amino acids of SEQ ID NO: 1, SEQ ID NO: 2, SEQ ID NO: 7, or SEQ ID NO: 8;
peptides comprising the sequence of SEQ ID NO: 3 or SEQ ID NO: 9; and
peptides comprising the entire sequence of SEQ ID NO: 1, SEQ ID NO: 2, SEQ ID NO: 7, or SEQ ID NO: 8.

38. The kit according to claim 34, wherein said one or more HCV antigenic peptides comprising in their sequence a sequence corresponding to at least 8 amino acids within the NS5a sequence are selected from the group consisting of:
peptides comprising at least 8 amino acids within in the 2209-2274 region, the 2250-2300 region or the 2275-2321 region of NS5a;
peptides comprising in their sequence at least 8 consecutive amino acids of SEQ ID NO: 4, SEQ ID NO: 5, SEQ ID NO: 10, or SEQ ID NO: 11;
peptides comprising the sequence of SEQ ID NO: 6 or SEQ ID NO: 12; and
peptides comprising the entire sequence of SEQ ID NO: 4, SEQ ID NO: 5, SEQ ID NO:I0, or SEQ ID NO: 11.

39. The kit according to claim 34, wherein said one or more peptides are peptides with overlapping peptide sequences.

40. The kit of claim 34, further comprising one or more reagents suitable for determining active HCV infection in a sample of a patient.

41. The kit of claim 40, wherein said one or more reagents suitable for determining active HCV infection in a sample comprise primers for the detection of HCV RNA levels for simultaneous or sequential testing and/or antibodies for the selective detection and quantification of HCV core antigen for simultaneous or sequential testing.

42. The kit according to claim 34, further comprising a secondary anti-Human IgG.

43. An in vitro method for predicting likelihood of the efficacy or outcome of a therapy against Hepatitis C Virus (HCV) infection in a patient, said method comprising: determining the presence in a serum or plasma sample of said patient of antibodies directed against the HCV antigens NS4a and/or NS5a; whereby absence of said antibodies against said NS4a antigens and/or NS5a antigens is indicative of a lower likelihood of benefiting of said therapy.

44. The method of claim 43, whereby determining the presence in a serum or plasma sample of said patient of antibodies directed against the HCV antigens NS4a and/or the NS5a is ensured using:
one or more HCV antigenic peptides comprising in their sequence at least 8 amino acids of the NS4a protein of HCV, and/or
one or more HCV antigenic peptides comprising in their sequence at least 8 amino acids of the NS5a protein of HCV.

45. The method of claim 43, wherein said therapy against Hepatitis C infection is based on interferon-alpha.

46. The method according to claim 44, wherein said determination of presence in a sample of said patient of antibodies directed against the HCV antigen NS4a and/or NS5a antigen is a quantitative determination.

47. The method according to claim 44, wherein said one or more HCV antigenic peptides are peptides comprising in their sequence at least 8 consecutive amino acids within the sequence of SEQ ID NO: 1, SEQ ID NO: 2, SEQ ID NO: 7, SEQ ID NO: 8, SEQ ID NO: 3, SEQ ID NO: 9. SEQ ID NO: 4, SEQ ID NO: 5, SEQ ID NO: 10, or SEQ ID NO: 11.

48. The method according to claim 44, wherein said one or more HCV antigenic peptides are peptides with overlapping peptide sequences.

49. The method according to claim 44, further comprising the step of detecting the HCV titer in a sample of said patient.

50. The method of claim 49, wherein said step of detecting the HCV titer in a sample is ensured by detecting the level of HCV RNA in a serum or plasma sample or by detecting a level of HCV core antigen in a serum or plasma sample of said patient.

51. The method according to claim 49, wherein said the step of detecting the HCV titer in a sample of said patient is performed on a sample taken after about at least 4 days, preferably at least 6 days after the start of HCV therapy.

52. The method of claim 49, wherein said the step of detecting the HCV titer in a sample of said patient, is only performed on those patients which are NS4a and/or NS5a positive based on the outcome of the determination of the presence in a serum or plasma sample prior to treatment of said patient, of antibodies directed against the HCV antigens NS4a and/or NS5a.

53. The method according to claim 47, wherein the combined presence of antibodies directed against the HCV antigens NS4a and/or NS5a being below a threshold value and either:
a level of HCV RNA in said sample of said patient above 105 IU/ml, or
a level of HCV core antigen in said sample of said patient above 15 pg/ml is indicative of non-response to therapy.

54. The method of claim 43, wherein said HCV is of genotype 1.

55. An isolated peptide corresponding to an NS4a or NS5a antigen, selected from the group consisting of:
peptides with a length between 8 and 30 amino acids containing a sequence of at least 8 amino acids of a polypeptide represented by the group of SEQ ID NO: 1, 2, 3, and 4;
peptides represented by SEQ ID NO: 1, SEQ ID NO: 2, SEQ ID NO: 4, SEQ ID NO: 5. SEQ ID NO: 7, SEQ ID NO:8, SEQ ID NO:10, and SEQ ID NO: 11;
peptides with a length between 8 and 30 amino acids comprising the sequence represented by SEQ ID NO: 4, SEQ ID NO: 6, SEQ ID NO: 10, or SEQ ID NO: 12; and
peptides selected from the group consisting of the peptides represented by SEQ ID NO: 3, SEQ ID NO: 6, SEQ ID NO: 9, and SEQ ID NO: 12.


SEQUENCE LISTING (12 sequences)

SEQ ID NO: 1 - protein, 20 aa, artificial
  Amino acid 1688-1707 of HCV non-structural protein NS4Aa Genotype 1a
LSGKPAIIPDREVLYREFDE

SEQ ID NO: 2 - protein, 20 aa, artificial
  Amino acid 1700-1719 of HCV non-structural protein NS4Aa Genotype 1a
VLYREFDEMEECSQHLPYIE

SEQ ID NO: 3 - protein, 8 aa, artificial
  Amino acid 1700-1707 of HCV non-structural protein NS4Aa Genotype 1a
VLYREFDE

SEQ ID NO: 4 - protein, 20 aa, artificial
  Amino acid 2263-2282 of HCV non-structural protein NS5 Genotype 1a
EDEREISVPAEILRKSRRFA

SEQ ID NO: 5 - protein, 20 aa, artificial
  Amino acid 2275-2294 of HCV non-structural protein NS5 Genotype 1a
LRKSRRFAQALPVWARPDYN

SEQ ID NO: 6 - protein, 8 aa, artificial
  Amino acid 2275-2282 of HCV non-structural protein NS5 Genotype 1a
LRKSRRFA

SEQ ID NO: 7 - protein, 20 aa, artificial
  Amino acid 1688-1707 of HCV non-structural protein NS4Aa Genotype 1b
LSGRPAVIPDREVLYQEFDE

SEQ ID NO: 8 - protein, 20 aa, artificial
  Amino acid 1700-1719 of HCV non-structural protein NS4Aa Genotype 1b
VLYQEFDEMEECASHLPYIE

SEQ ID NO: 9 - protein, 8 aa, artificial
  Amino acid 1700-1707 of HCV non-structural protein NS4Aa Genotype 1b
VLYQEFDE

SEQ ID NO: 10 - protein, 20 aa, artificial
  Amino acid 2263-2282 of HCV non-structural protein NS5 Genotype 1b
EDEREISVPAEILRKPRKFP

SEQ ID NO: 11 - protein, 20 aa, artificial
  Amino acid 2275-2294 of HCV non-structural protein NS5 Genotype 1b
LRKPRKFPPALPIWARPDYN

SEQ ID NO: 12 - protein, 8 aa, artificial
  Amino acid 2275-2282 of HCV non-structural protein NS5 Genotype 1b
LRKPRKFP